(19) United States
(12) Patent Application Publication
(10) Pub. No.: US 2012/0123270 A1
(43) Pub. Date: May 17, 2012

(54) DEVICE WITH INTEGRATED ULTRASOUND TRANSDUCERS AND FLOW SENSOR

(75) Inventors: Mareike Klee, Eindhoven (NL); Christianus Martinus Van Heesch, Eindhoven (NL); Peter Dirksen, Eindhoven (NL); Ruediger Mauczok, Eindhoven (NL); Nico Maris Adriaan De Wild, Eindhoven (NL)

(73) Assignee: KONINKLIJKE PHILIPS ELECTRONICS N.V., EINDHOVEN (NL)

(21) Appl. No.: 13/384,879

(22) PCT Filed: Jul. 26, 2010
(86) PCT No.: PCT/IB2010/053377

§ 371 Date: Jan. 19, 2012

(30) Foreign Application Priority Data
Jul. 29, 2009 (EP) 09166676.8

(51) Int. Cl. A61B 8/00

(52) U.S. Cl. 600/453

(57) ABSTRACT

The invention proposes to equip the tip of a surgical instrument such as a needle or catheter or any other instrument with an ultrasound transducer array to measure flow just in front of the tip by means of time and frequency differences between the sent and received pulses. Since no image is required, only a few transducer elements are required. The transducer elements generate a pressure pulses in specific directions and receives its echo's without the use of imaging techniques and complex driving electronics. Using the frequency shift and time delay of the received signals the proximity and lateral direction of the blood flow may be detected, thus identifying blood vessels.

### FIELD OF THE INVENTION

The present invention relates to an interventional system including a miniaturized device with ultrasound transducers and a processing means. Further, the invention relates to computer software for controlling the interventional system.

### BACKGROUND OF THE INVENTION

Surgical procedures are getting more and more minimally invasive. As a consequence, surgeons as well as radiologist or cardiologist do not see the target area, they need to work on. In order to navigate and perform the surgery on the correct place, typically for minimally invasive procedures instruments such as surgical tools, catheters, needles, and scopes and intra-operative imaging techniques are used such as echoscopy, X-ray and pre-operative imaging with CT and MRI-scans.

Furthermore, neurosurgery requires precise navigation of needles into the brain while avoiding damaging blood vessels inside the brain. Pre-operative images are often not enough, since the brain shifts its position when the skull is opened.

Today traditional ultrasound transducers based on ceramic or single crystal piezoelectric elements are used on for example catheters or guidewires for imaging purposes. These transducers consist of linear arrays to make 2D images. Using echoscopy the flow of blood is identified by image recognition, Doppler shift and/or speckle tracking with these ultrasound devices.

Integrated micro-machined ultrasound transducers (MUT) such as capacitive micro-machined ultrasound transducers (cMUT) and piezo micro-machined ultrasound transducers (pMUT) are new technologies to realize ultrasound transducer in Si based technologies. They are considered as a cheaper alternative to the existing piezoceramic transducers.

### SUMMARY OF THE INVENTION

It is an object of the invention to provide an interventional system and computer software for controlling the same, by means of which fluidic motion can be better detected.

This object is solved by the subject matter of the respective independent claims. Further exemplary embodiments are described in the respective depend claims.

Generally, an interventional system according to the invention comprises a miniaturized device and a processing means. The device comprises an array of ultrasound transducers located at a tip portion of an instrument such as a needle. The processing means is connected with the array of ultrasound transducers and is adapted to measure a frequency shift and a time delay between data sent and received by the ultrasound transducers.

In other words, the invention proposes to equip the tip of a needle or catheter with an array of ultrasound transducers to measure flow just in front of the tip by means of time and frequency differences between the sent and received pulses. Since no image is required, only a few transducer elements are required. The transducer elements generate an ultrasound pulse and receive its echo without the use of imaging techniques and complex driving electronics. Therefore, a limited amount of angles is required. Using the frequency shift and time delay of the received signals the proximity and lateral direction of the blood flow may be detected, thus identifying blood vessels.

It will be understood that the ultrasound transducers may be traditional transducers based on ceramic or single crystal piezoelectric elements, or may be MUTs produced in Si-technology, such as cMUTs or pMUTs.

Further, the ultrasound transducers may emit one pulse or a series of pulses, i.e. a burst. An ultrasound burst may consist of several pulses, namely between 1 and 10 pulses or between a few pulses as for example 10 or more and a plurality of pulses as for example 40. These pulses may be emitted omni-directional or in predefined directions, or may just mainly in one direction, i.e. having a small angle.

According to an embodiment of the invention, the processing means may be at least partially integrated in the device. The processing means may be divided in several processing sub-units each of which may be specialized to perform specific data processing steps or may be specialized to drive the ultrasound transducers according to the intended emitting characteristics. The processing sub-units may therefore be connected to each other in series or in parallel or a combination of said. As an example, one of said processing sub-units may be integrated in the device, i.e. may be applied in a separate chip which may be subsequently located in or at the device, especially in or at the tip portion of the device.

An advantage of that is a shortening of cables between each of the ultrasound transducers and the processing means. Since long cables have a negative influence on the quality of the signals transmitted, such a shortening will result in a more precise data received by the processing means. For example the heights and distances of several peeks sent from the transducers to the processing means may be measured more accurately.

Furthermore, the processing means may be integrally formed with the array of transducers. For example, in case the transducers are formed in an Si-substrate, driving electronics or processing circuits may also be realized in this Si-substrate, so that the array of ultrasound transducers together with a processing sub-unit may be located together at the tip portion of the device. Therefore, the array of ultrasound transducers may be processed on a separate carrier, which may also include a processing sub-unit and may be mounted at the tip portion of the device.

To avoid unintended effects on surrounding tissue, the array of ultrasound transducers may be covered with a biocompatible protection layer for example from parylene or any other organic or inorganic coating.

On the other hand, the complete tip portion, or even the complete device, including an array of ultrasound transducers as well as a processing sub-unit may be realized in an Si-substrate.

By way of this, a manufacturing process for a device according to the invention may have fewer steps which may result in a cheaper product. Further, the lengths of the signal path may be as short as possible.

In another embodiment, the processing sub-unit may provide for an amplification of the signals received from the ultrasound transducers. Also this will result in a higher accuracy of the processed data.

To influence the direction to which an ultrasound burst will be send, the array of ultrasound transducers may be arranged in form of a circle or in lines, or may be located on the surface of the bevel of the device or may be located at a circumferential surface of the device, wherein also a combination of both the bevel and circumferential surfaces may be advantageous depending on the intended application.

Based on a measuring of a frequency shift and a time delay between sent and received data, the processing means may be able to determine a spatial orientation and a distance of a fluid flow relative to the tip portion of the device, assuming that the position of the array on the tip portion is well known.

Such information may help an user (for example a physician) to first of all detect or recognize a fluid flow in the vicinity of the tip portion of the device, and thus to avoid injuring the vessel of the fluid flow.

It is noted that the device may be, on the one hand, a biopsy needle, a canula, or a trocar or, on the other hand, might also be a catheter, a guide wire, a scope or any other surgical instrument adapted to receive the device.

According to another embodiment of the invention, the interventional system may comprise a monitor and may also comprise an imaging device. The imaging device may be used for a non-invasive overview over the area of investigation or treatment. Such an imaging device may be an X-ray device like a fixed or mobile C-arm, a computer tomography device including a gantry, a magnet resonance tomography device, or an ultrasound device. Since it is sometimes difficult to detect blood vessels by one of the mentioned imaging devices without any contrast agent, the Ultrasound transducers together with the processing means may provide for additional information with respect to such vessels. The monitor which may be part of the imaging device may be used to visualize the data coming from the processing means as well as the image data from the imaging device.

In another embodiment of the invention also a larger number of MUT arrays as linear arrays or 2D arrays may be realized. The arrays mounted on a catheter or on a needle can be used during minimally invasive surgery procedures e.g. neurosurgery procedures to realize along forward or sideward looking ultrasound images to support positioning of the needle or catheter. The same ultrasound transducer arrays can also be used to determine along the time of flight of the transmit and receive signal as well as the frequency shift due to Doppler effect the blood flow and flow direction in blood vessels. The detected flow can for example be used to detect a vessel ahead or aside of the needle or catheter. This information is used in addition to the imaging to guide the needle or catheter and prevent damage of the vessel.

The invention relates also to a computer program for a processing device, such that a kind of a method might be executed on an appropriate system. The computer program is preferably loaded into a working memory of a data processor. The data processor is thus equipped to carry out the steps according to the invention. Further, the invention relates to a computer readable medium, such as a CD-Rom, at which the computer program may be stored. However, the computer program may also be presented over a network like the worldwide web and can be downloaded into the working memory of a data processor from such a network.

According to the invention, the computer software may cause an interventional system according to the invention to send an ultrasound pulse by means of ultrasound transducers, to record data received by the ultrasound transducers, and to measure a frequency shift and a time delay between the sent pulse and received data. Furthermore, the software may cause the interventional system to determine a spatial orientation and a distance between a fluid flow and the ultrasound transducers based on the measured frequency shift and time delay.

Finally, the computer software may cause the interventional system to visualize the processed data on a monitor, wherein also the image data received from an imaging device may be illustrated on said monitor.

It has to be noted that embodiments of the invention are described with reference to different subject matters. In particular, some embodiments are described with reference to method type claims whereas other embodiments are described with reference to apparatus type claims. However, a person skilled in the art will gather from the above and the following description that, unless other notified, in addition to any combination of features belonging to one type of subject matter also any combination between features relating to different subject matters is considered to be disclosed with this application.

The aspects defined above and further aspects, features and advantages of the present invention can also be derived from the examples of embodiments to be described hereinafter and are explained with reference to examples of embodiments. The invention will be described in more detail hereinafter with reference to examples of embodiments but to which the invention is not limited.

### BRIEF DESCRIPTION OF THE DRAWINGS

FIG. 1 shows an interventional system according to the invention.

FIG. 2a shows a first exemplary embodiment of an array of ultrasound transducers, wherein FIG. 2b also shows such an array relative to a fluid flow in a vessel.

FIG. 3 shows a tip portion of a device according to the invention including a second exemplary embodiment of an array of ultrasound transducers.

FIG. 4 shows a tip portion of a device according to the invention including a third exemplary embodiment of an array of ultrasound transducers.

FIG. 5 shows a tip portion of a device according to the invention including a fourth exemplary embodiment of an array of ultrasound transducers.

FIG. 6 illustrates examples of frequency shift and time delay.

FIG. 7 is an illustration of an interventional system according to the invention further including an imaging device and a monitor.

FIG. 8 is a flow chart of steps performed by an interventional system caused by computer software, according to the invention.

The illustration in the drawings is schematically only and not to scale. It is noted in different figures, similar elements are provided with the same reference signs.

### DETAILED DESCRIPTION OF EMBODIMENTS

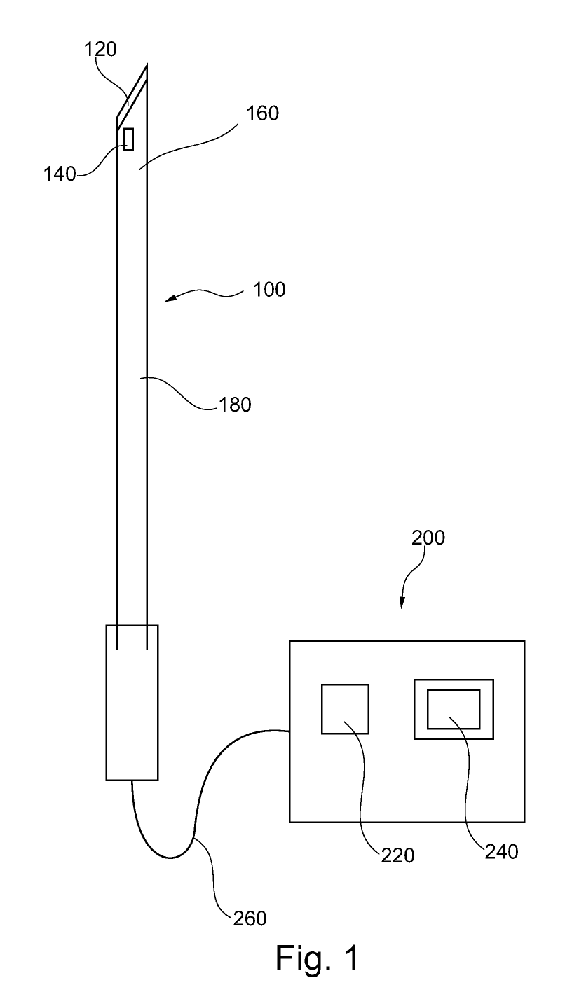

As illustrated in FIG. 1, a device 100 as part of a system according to an embodiment of the invention, comprises a shaft 180 at the distal end of which a tip portion 160 is located. At the tip portion 160, an array of ultrasound transducers 120 is arranged, wherein the array in this example is arranged on the surface formed by a bevel of the tip portion of the device. Additionally, a processing unit 140 is shown near the array 120 and in the tip portion of the device 100. The location of the processing unit 140 in FIG. 1 gives an example. The processing unit 140 can also be located at any other position e.g. directly underneath the ultrasound transducer 120 or at the end of the shaft 180.

Further shown in FIG. 1 is a console 200 including processing unit 220 and a monitor 240. The console 200 is connected with the device 100 by means of a cable 260. According to this exemplary embodiment, the processing unit 220 in the console 200 may via the cable 260 trigger the array of ultrasound transducers 120 to send an ultrasound pulse, wherein the reflections of said pulse coming back to the transducers, may be received by said transducers, will be converted into electrical signals, which then may be pre-processed by the processing unit 140. Subsequently, these pre-processed data will be transferred to the processing unit 220, further processed by said processing unit, and may be depict on the monitor 240.

According to the invention, an ultrasound transducer array may be realized as a linear but preferentially circular array of only a few elements on the tip of a needle, a catheter, or any medical device used to enter the body, to detect flows in a remote way. Without imaging being necessary, an ultrasound pulse can be sent and received in by the transducer array. Using the time and frequency differences between the sent and received pulses the flow direction and speed can be determined just in front of the catheter or needle, without using any imaging technique.

In an embodiment of the invention the few elements of the ultrasound transducer arrays are used to generate pressure pulses in several directions alternatively and to receive its echo without the use of imaging techniques and complex driving electronics.

Using the frequency shift and time delay of the received signals the proximity and lateral direction of the blood flow can be detected, thus identifying blood vessels.

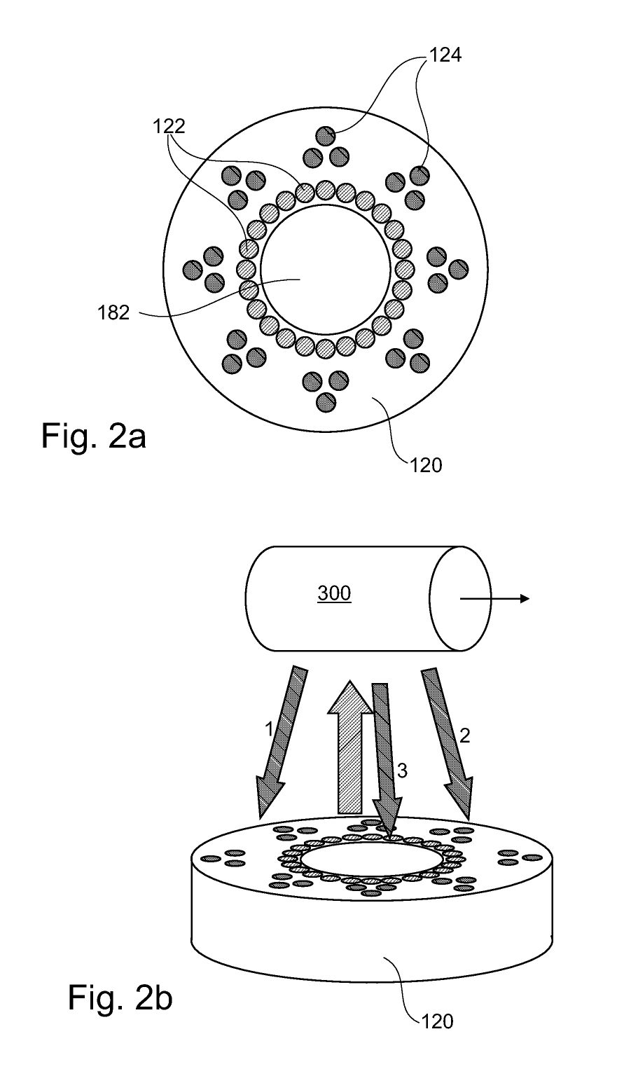

FIG. 2a shows a top view, and FIG. 2b an isometric view of a circular array of ultrasound transducers 120 according to an exemplary embodiment of the invention. In this configuration, it may be possible to arrange the array of transducers 120 around an opening of, for example, a through bore 182, which may be provided through the shaft of the device in axial direction.

In the examplary embodiment in FIGS. 2a and 2b, the sending MUTs 122 of the transducer are separated of the receiving MUTs 124. Regardless of the lateral orientation of the blood vessel 300 three different received signals can be identified. First received signals (indicated with ‘1’) the opposite direction of the flow direction have a high delay time and low frequency shift. Second received signals (indicated with ‘2’) along the flow direction have a high time delay and high frequency shift. Third received signals (indicated with ‘3’) orthogonal to the flow direction have a normal time delay (corresponding to the distance) and normal frequency shift (corresponding to the transmitted frequency).

In FIG. 2, eight groups of three MUTs 124 have been chosen as receiving MUTs. The more separated elements are chosen the higher the angular resolution of the direction of the flow will be. Higher numbers of elements per group result in a higher sensitivity.

On the other hand, the MUTs may be both transmitting and receiving elements. This results in a more complex driving electronics (equivalent to the imaging electronics) but reduces the size of the area of the device, making it more suitable for needle applications.

In any embodiment of the invention the driving electronics is either implemented in the Si-substrate either underneath the membranes or on top of the membranes or next to the membranes. The electronics can also be applied in a separate chip. To make the device ready for the application a biocompatible protection layer e.g. from parylene or any other organic or inorganic coating is applied.

Further, in any embodiment of the invention either a capacitive micro-machined ultrasound transducer (cMUT) or a piezoceramic micro-machined transducer (pMUT) can be used as either receiving or transmitting elements.

Different examples of implementation of the thin film transducers on top of a needle tip are shown in the following. Any other way of implementation on a needle or a catheter or scope or any other surgical instrument is also possible.

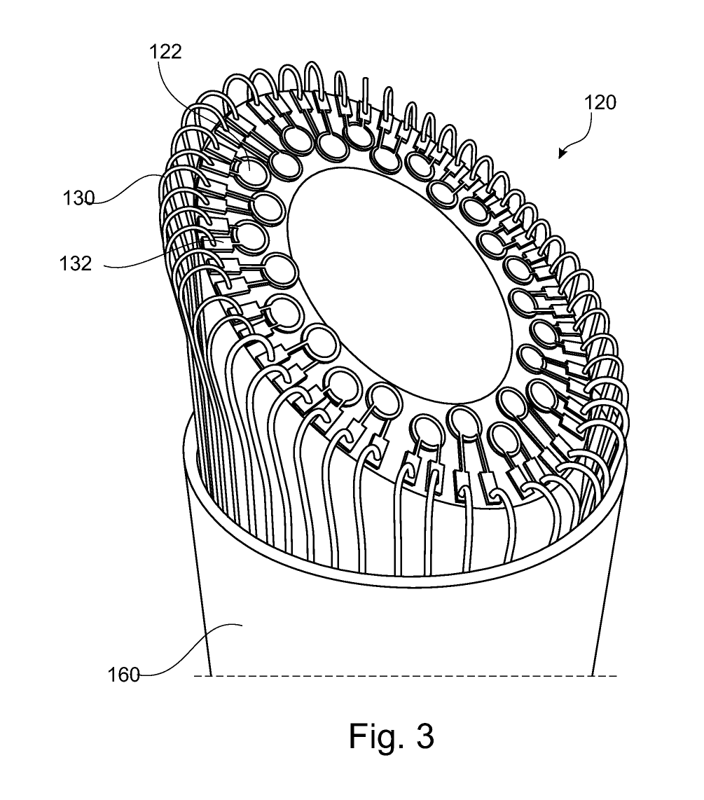

In FIG. 3 one example for the mounting of an array of ultrasound transducers 120 on a tip portion 160 of a device is given. Dependent on the device applied, which can be for example a needle which can have diameters of 1 mm up to 6 mm, the length of the tip portion 160 could be in the order of 1.5 mm up to 6 mm. The width of the tip portion 160 can be in the order of 1 mm up to 6 mm.

In FIG. 3 an example is shown for mounting thin film ultrasound transducers 122 such as capacitive micro-machined or piezoelectric micro-machined transducers on top of the bevel of a tip portion 160. The transducer array, which may be processed in Si technology have dimensions of the transducer, which are dependent on the operating frequency. For the circular membranes which are operating at several MHz up to 30 MHz the dimensions of the circular membranes are in the order of 20 μm up to 300 μm. Several elements 122 with this diameter are arranged on the needle tip. The single elements are contacted along bond pads 132, where in this case a wirebonding has been applied.

The wires 130 are transferred inside the needle and tip portion 160 to the processing unit. Besides the wire connection shown in FIG. 3 any other way of wired connection of the transducer elements is possible. For biocompatibility the transducers may be covered with a biocompatible material such as parylene.

In this example the transducers 122 which are processed on Si and arranged in a kind of circle on the needle tip. This opens the possibility to have in the center of the Si a hole, so that this transducer arrangement can be mounted also on a needle tip with an axial through bore.

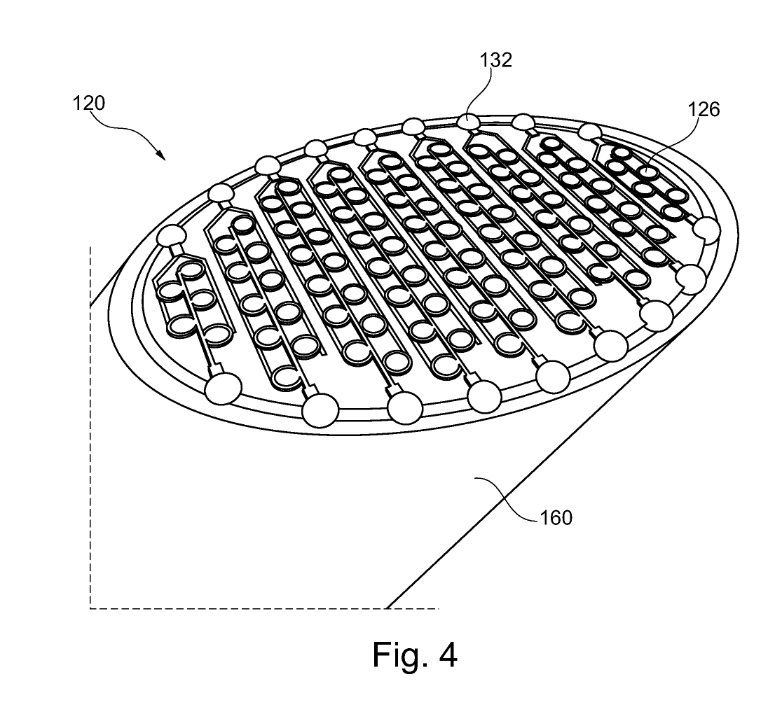

In FIG. 4 another exemplary embodiment is shown. Again a diameter for the needles of 1 mm up to 6 mm is used. The length of the tip portion 160 could be in the order of 1.5 mm up to 6 mm. The width of the needle tip can be in the order of 1 mm up to 6 mm. In FIG. 4 again capacitive micro-machined or piezoelectric micro-machined transducers 126 processed in Si technology are mounted. The dimensions of the transducer membranes are in the order of 20 μm up to e.g. 300 μm. Several elements 126 with this diameter are arranged on the needle tip. The elements are connected via interconnects among each other. At the edges flex interconnect within the needle is made using bump technology 132. Also here the needle tip may be covered with parylene.

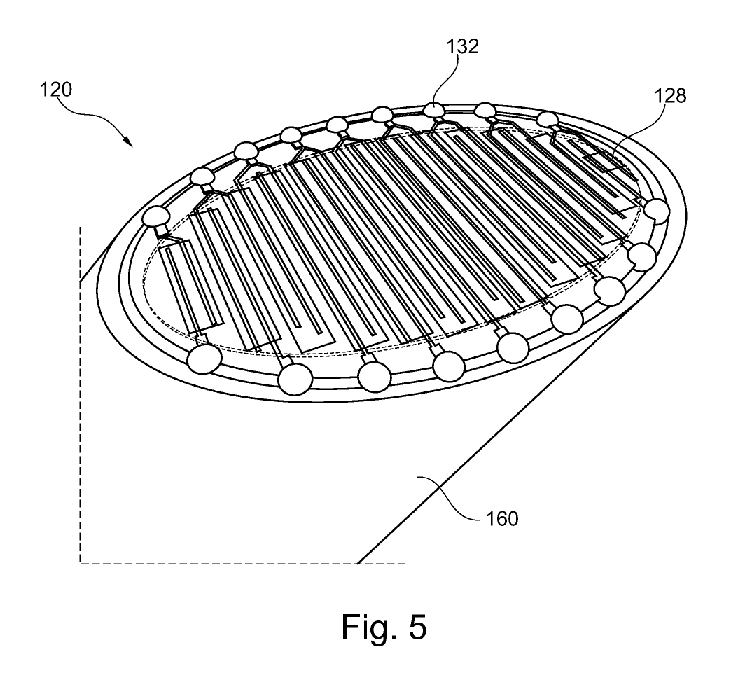

In another example, as shown in FIG. 5, a linear array of thin film ultrasound transducers 120 are processed on top of Si. The arrays are covering the dimensions of the tip portion 160 which can, dependent on the needle, have a length of 1.5 mm up to 6 mm and a width of 1 mm up to 6 mm. The arrays have a membrane width of 20 μm up to 300 μm, so that several elements 128 e.g. ten are mounted on the needle area. They are connected along metal interconnects to each other on the Si. The connection to the outside is realized either with wirebonding or bumping 132.

It should be mentioned here that the examples given in FIGS. 3-5 show the transducer arrays 120 mounted on the tip portion 160 of a device for a forward looking But it is also possible to mount a small amount of transducers, which are processed on top of Si on the side wall of the needles. Here the Si transducers are processed on a rigid or flexible carrier, which is mounted on the needle.

According to another embodiment of the invention, also a larger number of

MUT arrays as linear arrays or 2D arrays may be realized. The arrays mounted on a catheter or on a needle or any other surgical instrument can be used during minimally invasive surgery procedures e.g. neurosurgery procedures to realize along forward or sideward looking ultrasound images to support positioning of the needle or catheter. The same ultrasound transducer arrays can also be used to determine along the time of flight of the transmit and receive signal as well as the frequency shift due to Doppler effect the blood flow and flow direction in blood vessels. The detected flow can for example be used to detect a vessel ahead or aside of the needle or catheter. This information is used in addition to the imaging to guide the needle or catheter and prevent damage of the vessel.

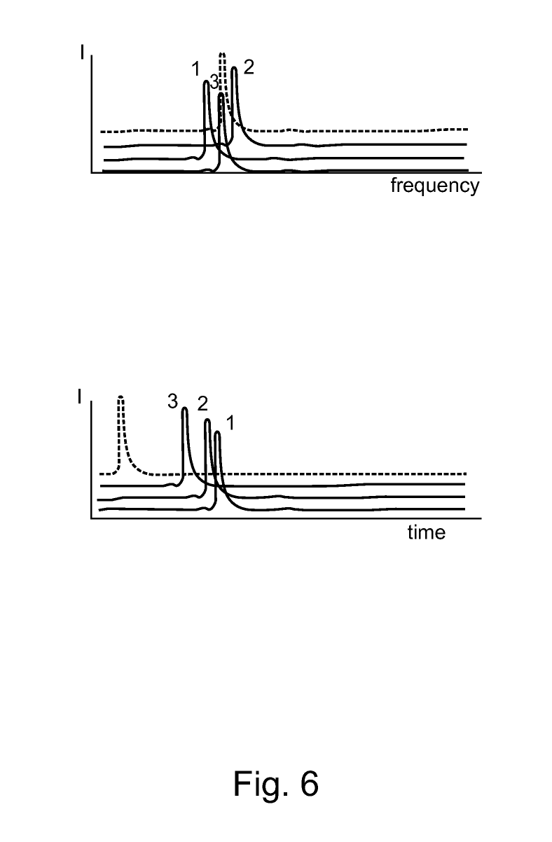

FIG. 6 show examples for frequency shift and time delay as measured by an interventional system according to the invention. Depict as dotted lines, a pulse sent by the transducers may provide for a reference signal in both diagrams. Relative to this reference signal, a first received signal (indicated with ‘1’) is sketched in having a high delay time and low frequency shift, which identifies an opposite direction of flow. A second received signal (indicated with ‘2’) is sketched in having a high time delay and high frequency shift, which identifies a direction along the flow direction. A third received signal (indicated with ‘3’) is sketched in having a normal time delay (corresponding to the distance) and normal frequency shift (corresponding to the transmitted frequency), which identifies a direction orthogonal to the flow direction.

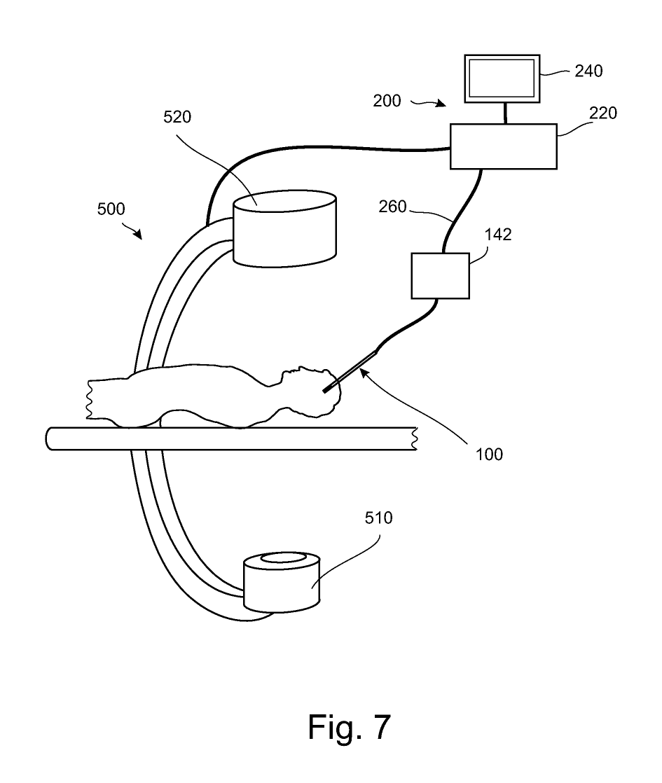

FIG. 7 shows an interventional system according to a further exemplary embodiment of the invention. The system comprises an elongated device 100, wherein an array of ultrasound transducers is located at the tip portion of the device, an imaging device 500 for assisting the coarse guidance, and a console 200. The imaging device 500 includes a radiation source 510 and a detector 520. In this example, the imaging device is a fixed C-arm. As another example, the imaging device may also be an ultrasound device, which provides intra-operative images.

The console 200 includes a processing unit 220 and a processing unit 142 for processing the signals coming from the imaging device 500 and from the device 100, and a monitor 240 for monitoring information for assisting the guidance of the biopsy device in a body.

As illustrated in FIG. 7, the interventional system comprises an image guided X-ray based intra-operative imaging system for guidance and a device comprising a sensor, i.e. an array of ultrasound transducers, which is connected with an processing unit 142, 220.

The system is able to follow the device from the incision to the target point by superimposing the information from the ultrasound transducers at the device on images from the imaging device and provide information about fluid flow at every point along the needle trajectory that is registered to the position inside the body of the patient. The region along the needle trajectory can be scanned (scan forward and scan aside) in order to provide indications on fluid flow existence in the vicinity of the tip of the device. Preferably in reconstructing where such a fluid flow is in front of the needle the X-ray data and the position information of the needle is actively used in the optical reconstruction of what is in front of the needle.

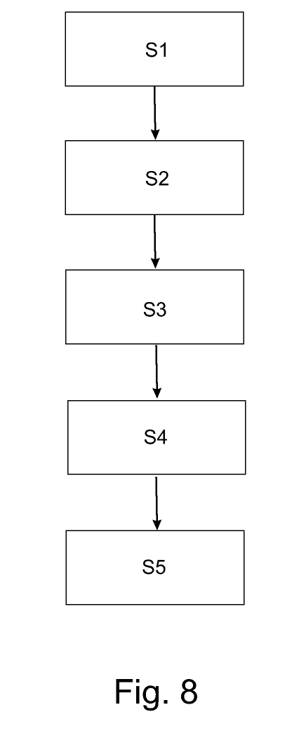

FIG. 8 is a flow chart, showing the steps of a method caused by computer software according to the invention when said software is executed on the processing unit of an interventional system as described above. It will be understood, that the steps described with respect to the method, are major steps, wherein these major steps might be differentiated or divided into several sub steps. Furthermore, there might be also sub steps between these major steps. Therefore, a sub step is only mentioned, if said step is important for the understanding of the principles of the method according to the invention.

In step S1, the ultrasound transducers at the tip portion of a device are driven so that an ultrasound burst is send out in a specific direction.

In step S2, reflected ultrasound vibrations are detected, i.e. received by the ultrasound transducers of the device omni-directionally or with a predetermined specific direction.

In step S3, a frequency shift and a time delay between the sent and the received ultrasound signals is measured by the processing unit of the interventional system.

In step S4, a special orientation and a distance between a fluid flow and the tip of the device may be determined.

Finally, in step S5, the information resulting from the preceding steps is visualized.

If for example the ultrasound burst is sent out not directly to the front but also to the side of the tip portion of the device, it may be advantageous to rotate the device about a few degrees and repeat the sending and receiving steps. This may improve the quality of the determination of the spatial orientation of a detected fluid flow, i.e. a vessel.

While the invention has been illustrated and described in detail in the drawings and foregoing description, such illustration and description are to be considered illustrative or exemplary and. not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

### LIST OF REFERENCE SIGNS

100 device

120 array of ultrasound transducers

122, 124 ultrasound transducer of circular array

126, 128 ultrasound transducer of linear array

130 wire

132 bond pads

140, 142 processing unit

160 tip portion

180 shaft

200 console

220 processing unit

240 monitor

260 cable

500 imaging device

510 radiation source

520 detector array

1. An interventional system comprising
a device (100) including a tip portion (160), wherein an array of ultrasound-transducers (120, 122, 124, 126, 128) is formed at the tip portion, and
a processing means (140, 142, 220) connected with the array of ultrasound-transducers, wherein the processing means is adapted to measure a frequency shift and a time delay between data sent and received by the array of ultrasound transducers.

2. The interventional system of claim 1, wherein the processing means comprises a processing sub-unit (140) which is integrated in the tip portion (160) of the device (100).

3. The interventional system of claim 1, wherein the processing sub-unit (140) is integrally formed with the array of ultrasound transducers (120, 122, 124, 126, 128).

4. The interventional system of claim 1, wherein the array of ultrasound transducers (122, 124) is arranged in form of a circle.

5. The interventional system of claim 1, wherein the array of ultrasound transducers (126, 128) is arranged in lines.

6. The interventional system of claim 1, wherein the array of ultrasound transducers (120, 122, 124, 126, 128) is processed on a separate cattier and is mounted at the tip portion (160) of the device (100).

7. The interventional system of claim 1, wherein the array of ultrasound transducers is covered with a biocompatible material.

8. The interventional system of claim 1, wherein the processing means (140, 142, 220) is adapted to determine the spatial orientation and distance of a fluid flow relative to the tip portion of the device (100), on the basis of the measured frequency shift and time delay.

9. The interventional system of claim 1, further comprising a monitor (240) for visualization of the processed data.

10. The interventional system of claim 9, further comprising an imaging device (500).

11. A device (100) for use in an interventional system according to claim 1.

12. A computer software when executed on a processing means (140, 142, 220), causing an interventional system according to claim 1:
to send an ultrasound pulse by means of ultrasound transducers (120, 122, 124, 126, 128),
to record data received by the ultrasound transducers, and
to measure a frequency shift and a time delay between the sent pulse and received data.

13. The computer software of claim 12, further causing the interventional system to determine a spatial orientation and a distance between a fluid flow and the ultrasound transducers (120, 122, 124, 126, 128).

14. The computer software of claim 12, causing an interventional system to visualize the processed data on a monitor (240).

15. The computer software of claim 12, causing an interventional system to visualize the data processed by the processing means together with the image data received from an imaging device (500).
